(19) United States
(12) Patent Application Publication
(10) Pub. No.: US 2012/0089195 A1
(43) Pub. Date: Apr. 12, 2012

(54) SYSTEM AND METHOD FOR SELF FILLING BONE SCREWS

(75) Inventors: Joseph W. Yedlicka, Indianapolis, IN (US); Robert A. Till, JR., Avon, IN (US)

(73) Assignee: OSTEO INNOVATIONS, LLC, Indianapolis, IN (US)

(21) Appl. No.: 13/276,149

(22) Filed: Oct. 18, 2011

Related U.S. Application Data
(63) Continuation of application No. PCT/US10/31697, filed on Apr. 20, 2010.
(60) Provisional application No. 61/170,688, filed on Apr. 20, 2009.

(51) Int. Cl. A61B 17/86; A61B 17/88

(52) U.S. Cl. 606/304

(57) ABSTRACT

A self filling autograft bone screw for stabilizing and fusing bones within a body of a patient. The self filling autograft bone screw includes an elongated body member, a lumen disposed within the elongated body member, a plurality of external threads, a cutting section, and at least one opening disposed along the length of the elongated body member. A system and method for inserting a bone screw into a bone of a patient's body includes advancing and positioning a self-filling bone screw including an elongated body member, a lumen disposed within the elongated body member, a plurality of external threads, a cutting section, and at least one opening disposed along the length of the elongated body member into a bone within a patient's body.

### CROSS-REFERENCE TO RELATED APPLICATIONS

This application claims the benefit of U.S. Provisional Application No. 61/170,688, filed on Apr. 20, 2009, the entire contents of which is incorporated herein by reference.

### TECHNICAL FIELD

The invention relates to systems and methods for spinal stabilization and fusion, and more particularly, to systems and methods for stabilizing and fusing facet joints within a body of a patient.

### BACKGROUND INFORMATION

The individual vertebrae in the spine of a body of a patient are joined to each other at three sites: the intervertebral disc and two facet joints. Each vertebra has an articulating surface (facet) on the left and right sides when joined with the articulating surfaces (facets) of the adjacent vertebrae. These articulating surfaces form facet joints. Each facet joint is a true synovial joint comprised of cartilaginous surfaces surrounded by a capsule of connective tissue. These joints contain synovial fluid which lubricates and nourishes the joints. The cartilaginous surfaces and synovial fluid allow the joints to move or articulate with each other.

Unfortunately, facet joints and intervertebral discs are commonly diseased, degenerated, or arthritic which can result in significant pain. This pain can be treated by stopping motion and stabilizing the diseased vertebral segment(s). Such treatment is typically known as fusion. Fusion involves fusing all three sites of articulation: the intervertebral disc space and the facet joints. Posterior (or facet joint) fusion can be accomplished by placement of pedicle screws and posterior rods or by direct facet joint fusion. These fusion procedures have traditionally involved open surgery, and more recently the trend has been toward minimally invasive and percutaneous procedures. Surgical procedures have been hampered by prolonged postoperative recovery, as well as considerable peri- and postoperative morbidity and mortality. Currently available screws are limited by screw “loosening” or “backing out”, particularly in osteoporotic bone.

Thus, there is a need for improved percutaneous instrumentation and techniques that result in safe, effective fusion and stabilization of facet joints as well as placement of pedicle screws with screw retention features. Also, there is a need for improved bone screws with screw retention features for other orthopedic/neurosurgical applications such as intramedullary rods, bone plating, and artificial joint placement requiring screws.

### SUMMARY OF THE INVENTION

According to one aspect, the invention relates to a self-filling autograft bone screw comprised of an elongated body member, a lumen disposed within the elongated body member, a plurality of external threads, a cutting section, and at least one opening disposed along the length of the elongated body member. The elongated body member has a proximal portion and a distal portion. The lumen extends from a proximal end of the elongated body member to a distal end of the elongated body member. A plurality of external threads extends from the proximal portion of the elongated body member to the distal portion of the elongated body member. The plurality of external threads are adapted for anchoring the elongated body member within an internal portion of a bone within a patient's body. The cutting section is disposed at the distal end of the elongated body member. The cutting section is adapted to enable penetration of the bone screw into the internal portion of the bone and facilitate the insertion of fragments into the lumen resulting from the penetration of the bone screw into the internal portion of the bone. At least one opening is disposed along the length of the elongated body member. In addition, at least one opening is adapted for facilitating the re-growth of the fragments within the internal portion of the bone and anchoring of the elongated body member within the internal portion of the bone.

According to a second aspect, the invention relates to a screw system for inserting a bone screw into a bone of a patient's body comprised of an external fastening member, a self filling bone screw comprised of an elongated body member, a lumen disposed within the elongated body member, a plurality of external threads, a cutting section, and at least one opening disposed along the length of the elongated body member. The external fastening member is used to facilitate insertion of the self filling bone screw into the bone within the patient's body. The external fastening member includes at least one flute member disposed along the length of the external fastening member. The elongated body member of the self filling bone screw has a proximal portion and a distal portion. A lumen is disposed within the elongated body member. The lumen extends from a proximal end of the elongated body member to a distal end of the elongated body member. A plurality of external threads extend from the proximal portion to the distal portion. The plurality of external threads are adapted for anchoring the elongated body member within an internal portion of a bone within a patient's body. The cutting section is disposed at the distal end of the elongated body member. The cutting section is adapted to enable penetration of the bone screw into the internal portion of the bone and facilitate the insertion of fragments into the lumen resulting from the penetration of the bone screw into the internal portion of the bone. At least one opening is disposed along the length of the elongated body member. In addition, at least one opening is adapted for facilitating the re-growth of the fragments within the internal portion of the bone and anchoring of the elongated body member within the internal portion of the bone.

According to a third aspect, the invention relates to a method for inserting a bone screw into a bone of a patient's body. The method includes providing a self filling bone screw, such as one of the self filling bone screws described above, forming a hole within the bone, advancing the bone screw into the hole within the bone, and positioning the bone screw into the hole within the bone.

### BRIEF DESCRIPTION OF THE DRAWINGS

In the drawings, like reference characters generally refer to the same or similar parts throughout the different views. Also, the drawings are not necessarily to scale, emphasis instead generally being placed upon illustrating the principles of the invention.

FIG. 1 is a perspective view of the embodiment of a self filling autograft bone screw;

FIG. 2 is a perspective view of the self filling autograft bone screw including a washer member;

FIG. 3 is a perspective view of the self filling autograft bone screw including a wiper member;

FIG. 4 is a cross sectional view of the self filling autograft bone screw of FIG. 1;

FIG. 5 is an exploded perspective view of an external fastening member in use with the self filling autograft bone screw of FIG. 4;

FIG. 6 is a perspective view of an external fastening member in use with the self filling autograft bone screw of FIG. 1;

FIG. 7 is an exploded perspective view of the self filling autograft bone screw of FIG. 6;

FIG. 8 is perspective view of another embodiment of the self filling bone screw of FIG. 1;

FIG. 9 is perspective view of another embodiment of the self filling bone screw of FIG. 1;

FIG. 10 is perspective view of another embodiment of the self filling bone screw of FIG. 1;

FIG. 11 is perspective view of another embodiment of the self filling bone screw of FIG. 1;

FIG. 12 is cross sectional view of another embodiment of the self filling bone screw of FIG. 11;

FIG. 13 is perspective view of another embodiment of the self filling bone screw of FIG. 1;

FIG. 14 is perspective view of another embodiment of the self filling bone screw of FIG. 1;

FIG. 15 is perspective view of another embodiment of the self filling bone screw of FIG. 1;

FIG. 16 is cross sectional view of another embodiment of the self filling bone screw of FIG. 15;

FIG. 17 is a perspective view of another embodiment of the external fastening member and the self filling bone screw of FIG. 1;

FIG. 18 is a perspective view of the external fastening member attached to the self filling bone screw of FIG. 1;

FIG. 19 is a perspective view of the external fastening member attached to the self filling bone screw of FIG. 1;

FIG. 20 is a perspective view of the external fastening member attached to the self filling bone screw of FIG. 1;

FIG. 21 is a perspective view of another embodiment of the external fastening member attached to the self filling bone screw of FIG. 17;

FIG. 22 is an exploded perspective view of the external fastening member attached to the self filling bone screw of FIG. 21;

FIG. 23 is an exploded perspective view of the external fastening member and the self filling bone screw of FIG. 22;

FIG. 24 is a cross sectional view of the external fastening member attached to the self filling bone screw of FIG. 21;

FIG. 25 is an exploded cross sectional view of the external fastening member attached to the self filling bone screw of FIG. 21;

FIG. 26 is a perspective view of another embodiment of the external fastening member and the self filling bone screw of FIG. 1;

FIG. 27 is a perspective view of the external fastening member attached to the self filling bone screw of FIG. 26;

FIG. 28 is another perspective view of the external fastening member attached to the self filling bone screw of FIG. 26; and

FIG. 29 is an exploded perspective view of the external fastening member of FIG. 26.

### DESCRIPTION

In general, the invention relates to self filling bone screws which may be used for applications such as spinal stabilization and fusion, intramedullary (IM) rods, joint implants, plating, or any other orthopedic/neurosurgical application where such screws would be desirable. Such bone screws may be used with a radio-lucent, off-angle, motorized drill system. The bone screws may also be placed with an insertion tool and manual drive handle or a standard power drill.

The self filling autograft bone screw is designed with distal cutting edges which direct native cortical and cancellous bone and marrow into a lumen of the hollow screw. At least one opening is provided along the screw shaft to allow bone to grow into and out of the screw in order to form a lattice-work of bone criss-crossing through the screw and in order have the screw incorporated into the bone. Thus, the screw forms its own internal bone graft (autograft) with its resultant osteogenesis, osteoconduction, and osteoinduction properties. This allows the autograft screw to provide immediate fixation (due to the screw) as well as long-term fixation (due to the screw and bony fusion). It may also be used with bone morphogenetic protein to facilitate bone growth.

The self filling autograft bone screw also has a self-tapping and thread cutting design. A floating washer member is provided to allow the washer to pivot on the spherical undersurface of the proximal end of the screw to make better contact with angled bony surfaces with resultant improved holding power. A wiper member is also provided in the screw head which strips bone material from an external fastening member, such as a guide drill, when the guide drill is removed to maximize the amount of autograft in the screw. A stepped shank design, (smaller diameter at the distal end in contrast to a greater diameter at the proximal end), may be used to provide greater grip and strength in the endosteal (undersurface) area of the bone. A one-step delivery device and system is also provided which facilitates placement of the screw. This one-step delivery device may be driven manually or with a powered driver.

Percutaneous insertion of the self-filling bone screw may be accomplished with an insertion tool and manual drive handle. The bone screw may be positioned in an insertion tool which has spring catches designed to hold the screw securely in the insertion tool. A long drill bit may be placed through the insertion tool and screw. The assembly may then placed through a small incision to the bone (e.g. pedicle, facet joint, etc). The guide drill and screw are then advanced into the bone—this may be done with a manual turning handle, standard drill, or radio-lucent, off-angle drill. Once the guide drill has entered the bone and provided small initial pilot hole, the handle releases the guide drill and engages the screw drive. The screw is then advanced to its final depth. The drive handle (or drill) is removed, the sleeve retracted up along the insertion tool body releasing the spring catches holding the screw in the insertion tool. Once the catches are released, the screw is left in place and the insertion tool is removed.

Percutaneous placement of the self-filling bone screw may also utilize bone morphogenetic protein (BMP) to facilitate bone growth. The BMP wafer or putty may be placed inside the screw prior to placement.

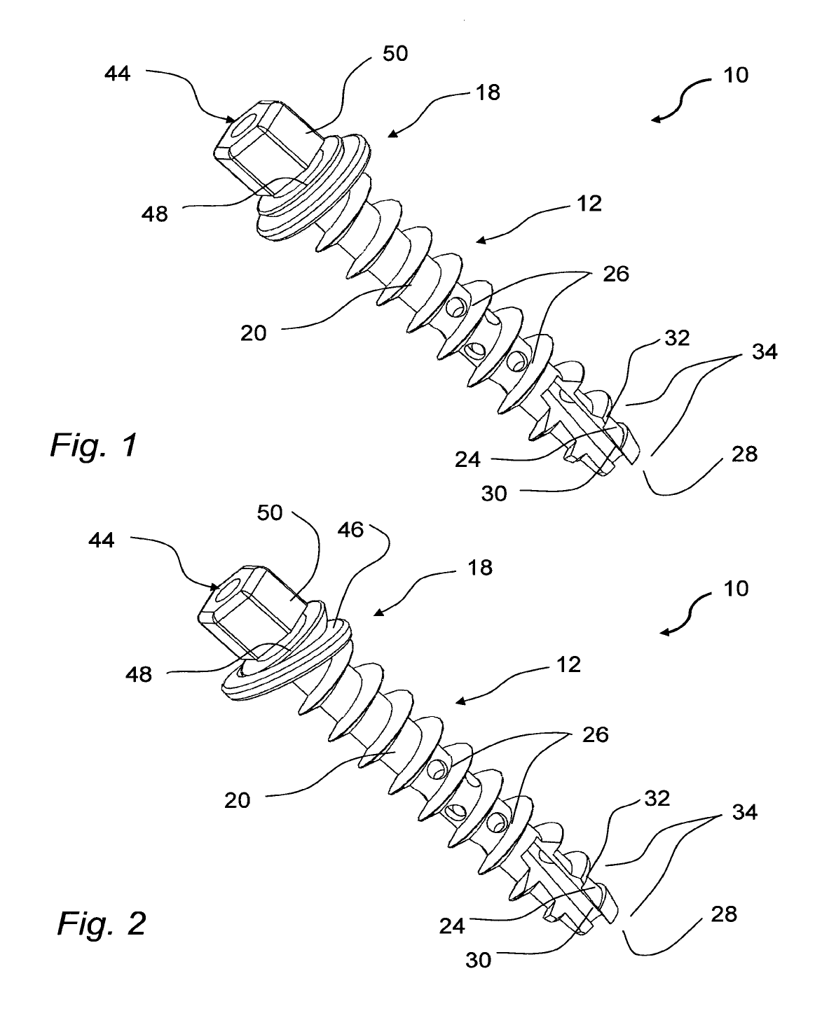

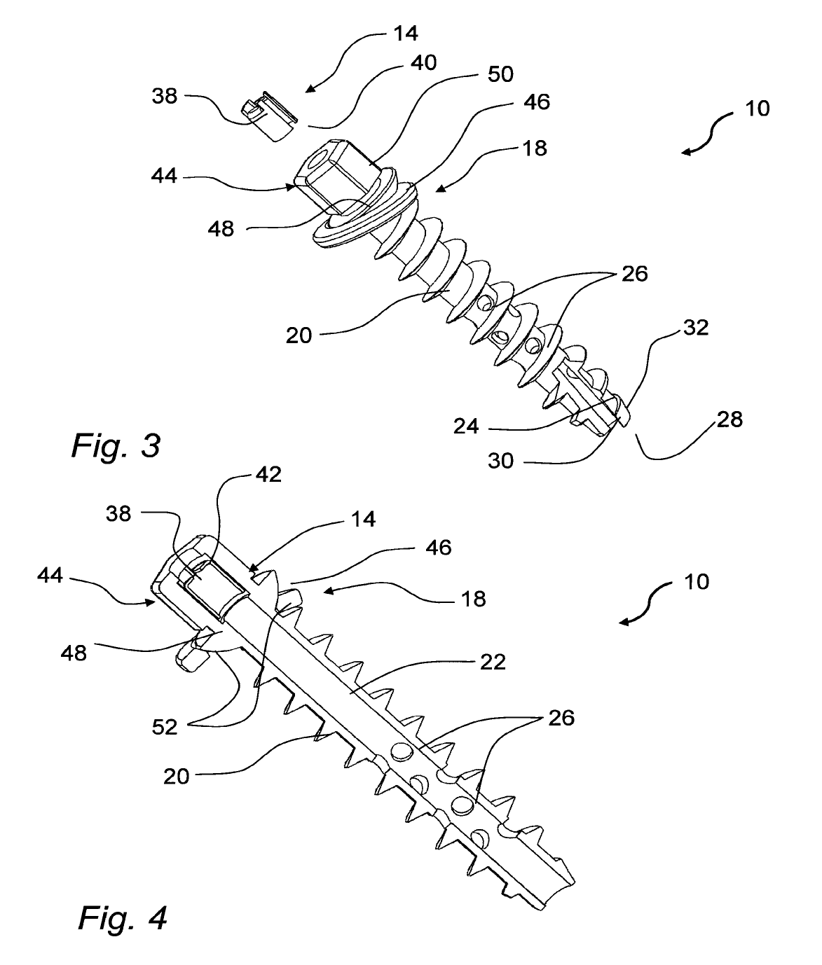

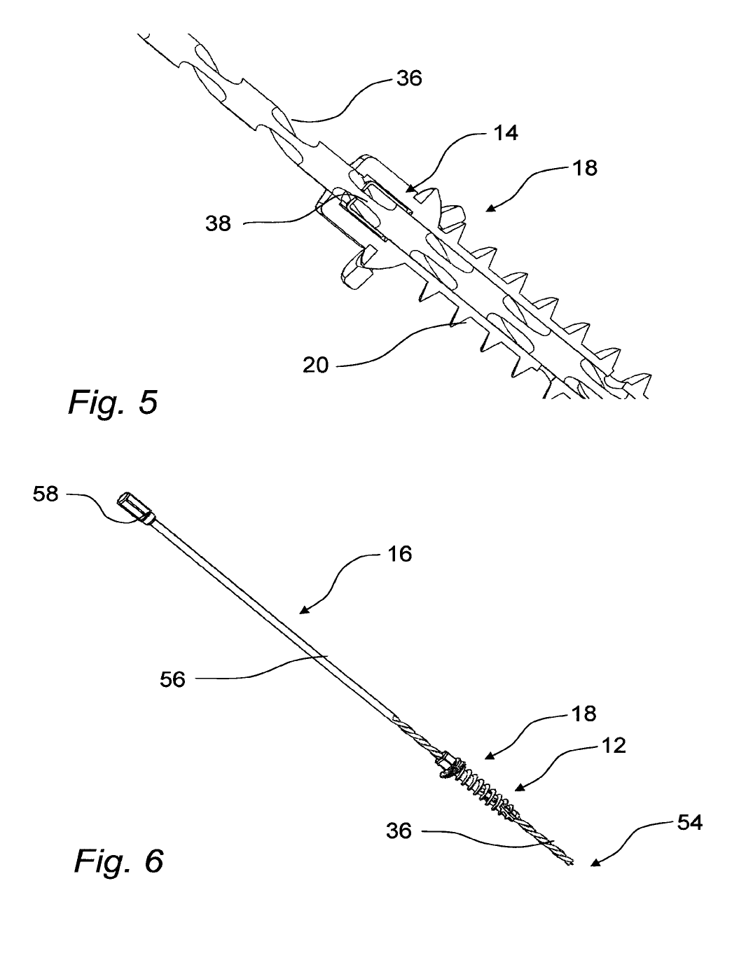

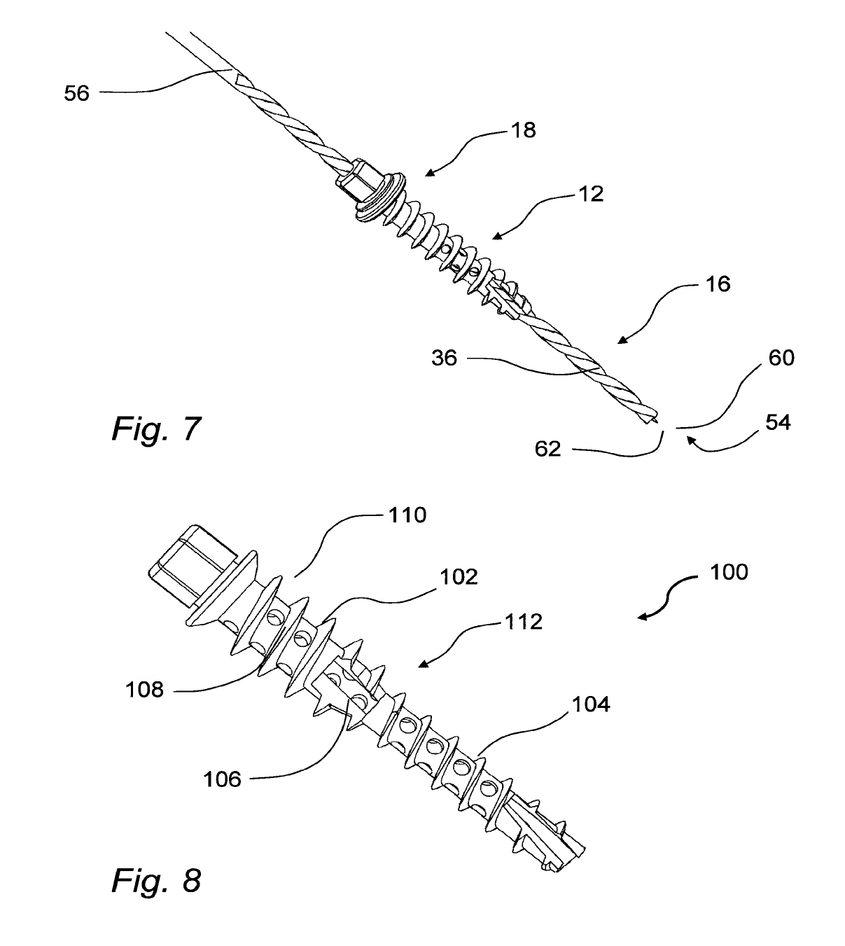

Referring to FIGS. 1 through 7, in one embodiment according to the invention, a self filling autograft screw 10 includes an elongated screw body member 12 having a proximal portion and a distal portion. The proximal portion of the elongated screw body member includes a washer 18. The elongated screw body member 12 includes a lumen passage 22 which extends from a proximal end of the elongated body member 12 to a distal end of the elongated body member 12. The screw 10 further includes a plurality of external threads 20 which extend from the proximal portion of the elongated body member 12 to the distal portion of the elongated body member 12. The plurality of external threads 20 are adapted for anchoring the elongated body member 12 within an internal portion of a bone within a patient's body. The bone screw 10 further includes a cutting section 24 disposed at the distal end of the elongated body member 12. The cutting section 24 is adapted to enable penetration of the bone screw 10 into the internal portion of the bone and facilitate the insertion of fragments into the lumen 22 resulting from the penetration of the bone screw into the internal portion of the bone. At least one opening 26 is provided along the length of the elongated body member 12. The opening 26 is adapted for facilitating the re-growth of the fragments within the internal portion of the bone and anchoring of the elongated body member 12 within the internal portion of the bone.

The bone screw 10 may also consist of an elongated body member 12, a wiper member 14, and a washer member 18. The bone screw 10 may be inserted into and left in a bone to hold or affix objects. This can be either bone to bone or bone to an artificial construct. All parts of the bone screw 10 may be made from implant compatible materials.

The external threads 20 can be of various shapes, sizes, or pitches. The external threads 20 anchor the bone screw 10 into the bone. The bone screw 10 self fills the lumen passage 22 with fragments such as chips of bone, bone marrow, and blood as it is inserted into the bone by directing the fragments cut by the external fastening member, such as a guide drill 16, and the thread cutting section 24 at the distal tip of the elongated body member 12.

As the bone heals, bone re-growth occurs between the bone material inside the bone screw 10 and the bone material which is inserted through the various openings 26 in the elongated body member 12. The openings 26 can be of various shapes, sizes, quantities, and locations around the elongated body member 12. It is contemplated that the bone incorporated into the bone screw 10 will increase pullout force, resistance to loosening, and enhance the overall structural integrity of the surrounding bone.

The cutting section 24 consists of helical formed cutting surface at the distal end of the elongated body member 12 and a slot 30 that forms a cutting edge 32. The cutting edge 32 has an acute angle relative to the longitudinal axis of the elongated body member 12 and feeds cut bone fragments into the lumen passage 22. The slot 30 extends proximal through the threads 34 to a first full thread. This configuration allows the cutting edge 32 to cut threads into the bone as the bone screw 10 is advanced.

The lumen passage 22 is formed by a cylindrical bore with a diameter equal to or slightly larger than the diameter of the guide drill 16. The lumen passage 22 thus follows the guide drill 16 as the bone screw 10 is threaded into the bone. The openings 26 pass through the elongated body member 12 and allow new bone growth to connect the surrounding bone with the lumen passage 22. Fragments are deposited into the lumen passage 22 by scraping the material captured in flutes 36 of the guide drill 16 with the wiper 14.

The wiper 14 may be cylindrical in shape and have an inner diameter that is equal to or greater that the outer diameter of the guide drill 16. The wiper 14 has formed wiping features 38 that approximate the cross sectional shape of the flutes 36. The wiper 14 has a slit 40 along one side parallel to the center axis. The slit 40 allows the wiper 14 to be compressed and inserted into the lumen passage 22 of the elongated body member 12 to a position where the secondary cavity 42 has been formed to receive it. The secondary cavity 42 has an inner diameter that is larger than the outer diameter of the wiper 14 allowing it to spin freely within the elongated body member 12. In another embodiment, the wiper 14 may be disposed within a distal portion of a drive shaft of the guide drill 16. This configuration can also facilitate the removal of fragments to be deposited into the lumen passage 22.

As the guide drill 16 is rotated and extended into the bone forming a pilot hole, it collects the bone chips along the flutes 36. As the bone screw 10 is translated towards the distal tip of the guide drill 16, by either threading the bone screw 10 over the guide drill 16 or by retracting the guide drill 16 after the bone screw 10 has threaded into the bone, the wiping features 38 of the wiper 14 prevent fragments, such as bone chips, from leaving the lumen passage 22 or the elongated body member 12. When the guide drill 16 is fully retracted from the elongated body member 12, a significant portion of the bone chips will be left in the elongated body member 12.

Various coatings, such as hydrophobic, hydrophilic, or BMP, may be affixed to different surfaces of the elongated body member 12, wiper 14, and guide drill 16 to facilitate the translation of the bone fragment/blood products into the elongated body member 12 and to aid in promoting regenerative bone growth.

The proximal end of the screw body 12 contains a head section 44 which consists of a contact surface 46, a capture feature 48, and a drive structure 50. The contact surface 46 mates with the outer surface of the bone that the bone screw 10 is driven into. The surface can be flat, for example, relative to the axis of the bone screw 10, concave or convex. The outer diameter of the contact surface 46 defines the surface area supporting the axial loading experienced by the bone screw 10 in use. Alternatively, the washer 18 can be installed between the contact surface 46 and the outer surface of the bone.

In an embodiment, the washer 18 has a spherical depression 52 on the side that mates to the contact surface 46 of the bone screw 10 with a radius that matches that of the convex contact surface 46. The hole through the center of the washer 18 is larger that the diameter of the elongated body member 12 where it is located. This, along with the mating spherical contact surface 46 and depression 52, allows the washer 18 to float angularly about the head section 44. In addition, the ability for floating facilitates the washer 18 in making contact with the outer surface of the bone when that outer surface is angled relative to the axis of the elongated body member 12.

The capture feature 48 may be undercut in the drive structure 50. Alternatively, the capture feature 48 could be a depression in the wall of the drive structure 50. The capture feature 48 facilitates the installation of the bone screw 10 by providing a means to securely retain the bone screw 10 in the external fastening member until its desired release.

The drive structure 50 may be a standard external hex-shape that can transmit driving torque required to thread the bone screw 10 into the bone. This external hex-shape design aids in removing the screw in a subsequent surgical procedure. It is contemplated that alternate external and internal configurations of the drive structure 50 are possible.

The guide drill 16 consists of a cutting tip 54, flutes 36, body 56, and optionally a drive structure 58. The cutting tip 54 can take many shapes. In one embodiment, the cutting tip 54 has a sharp brad point 60 in the center and a flat cutting edge 62 radiating out towards the outer diameter. The flutes 36 can be designed in different shapes, such as circular, parabolic, or other. The flutes 36 may extend partway up the body 56. The proximal end of the guide drill 16 can also remain cylindrical allowing the use of a conventional Jacob's style chuck or ratchet, or have a drive structure 58 that has the same external hex shape as on the proximal end of the elongated body member 12.

Referring to FIG. 8, an alternative embodiment of the self filling autograft bone screw 10 of FIG. 1 is presented. A bone screw 100 is provided which includes an elongated body member 112 with a shank or thread root having a diameter that is larger at a proximal portion 102 than at a distal portion 104. The difference in diameter provides for a stronger elongated body member 112 that will bear greater stress while maintaining a smaller outer diameter where the bone may be smaller in cross section. Additional openings 108 and a second thread cutting section 106 may be added to accommodate a larger thread 110 that is incorporated into the elongated body member 112.

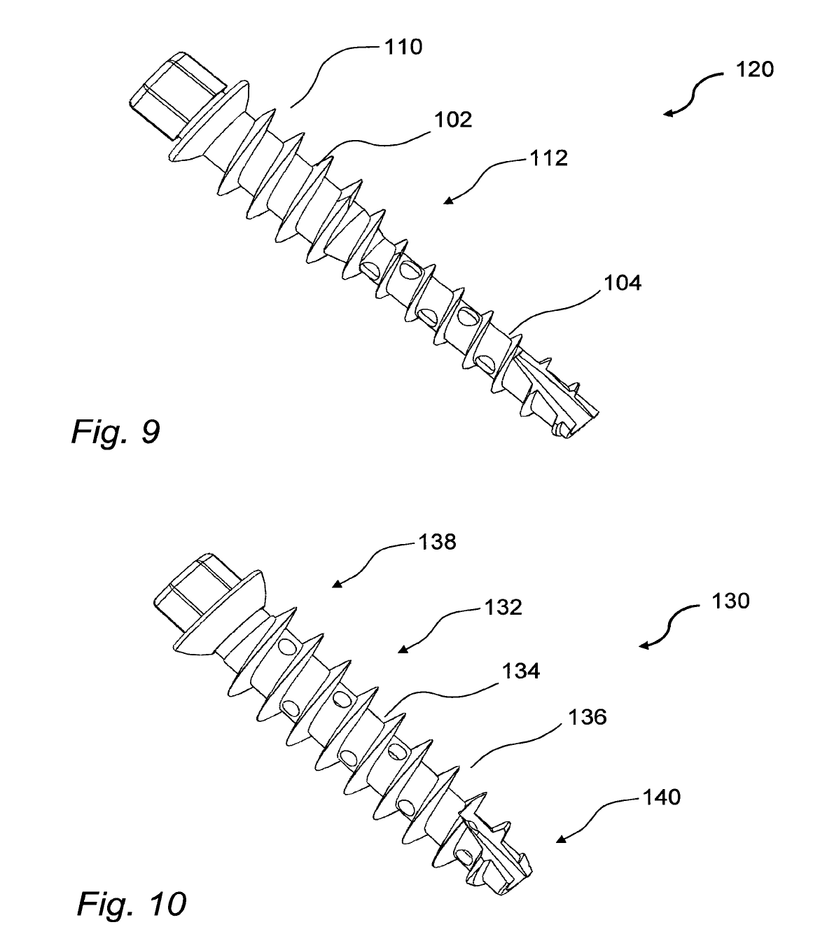

Referring to FIG. 9, an alternative embodiment of the self filling autograft bone screw 10 of FIG. 1 is presented. A bone screw 120 is provided in which the proximal portion 102 has a larger diameter relative to the distal portion 104 of the elongated body member 112. The distal portion 104 of the elongated body member 112 is free of additional openings 108 and the secondary thread cutting section 106. This design can improve the strength of proximal portion 102 of the elongated body member 112.

Referring to FIG. 10, an alternative embodiment of the self filling autograft bone screw 10 of FIG. 1 is presented. A bone screw 130 is provided in which an elongated body member 132 has a taper shank or root diameter 134 and tapered threads 136. This design can provide greater strength at the proximal portion of the shank and a smaller, less invasive cross section at a distal portion 140.

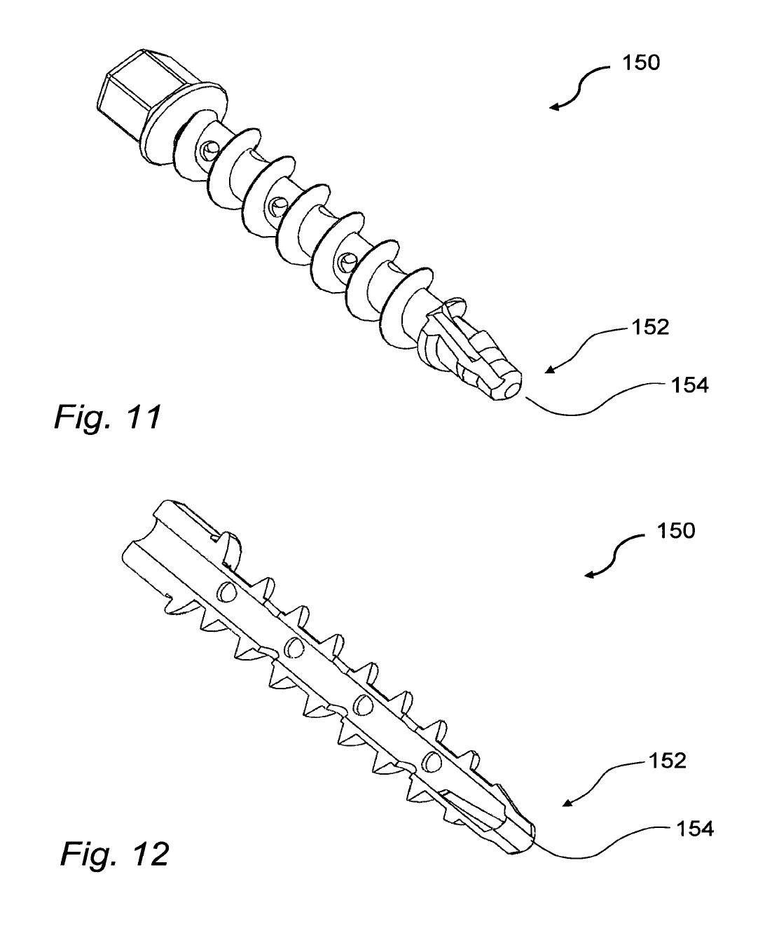

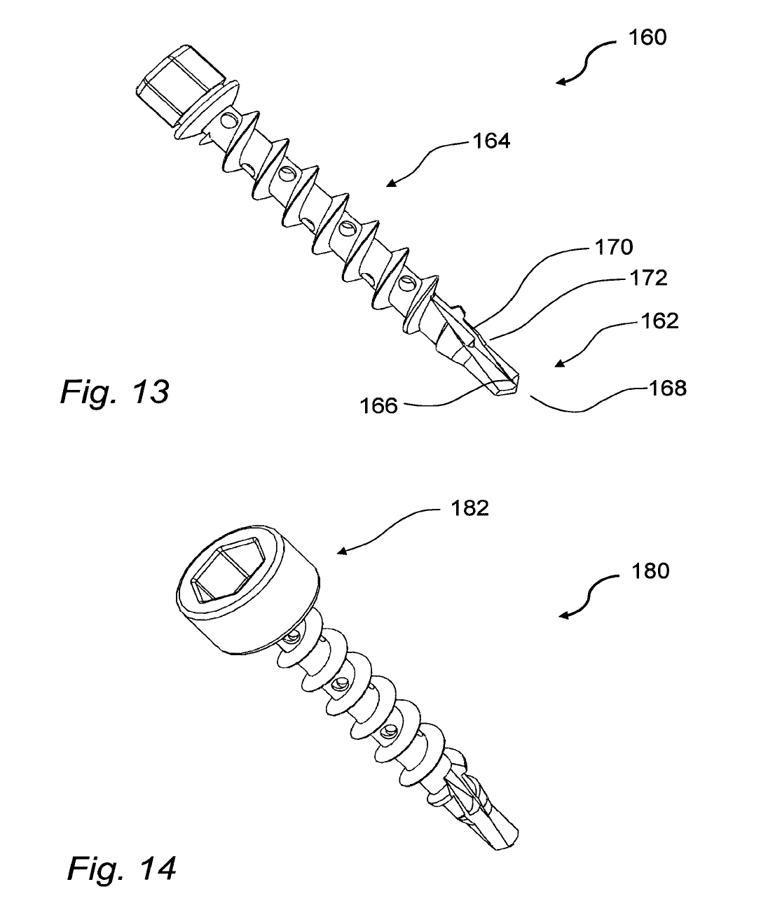

Referring to FIGS. 11 through 14, alternative embodiments of the self filling bone screw 10 of FIG. 1 are presented. Each of the alternative embodiments may be used without an external fastening member, such as guide drill 16.

A bone screw 150 is provided which has a small diameter cylindrical cut 154 through a drill point 152. This design allows the bone screw 150 to follow a standard K-wire into a pilot hole previously formed in the bone. A bone screw 160 is provided having a drill point 162 added to the distal end of an elongated body member 164. The drill point 162 has a standard cutting edge 168 and double flutes 166. In addition, the drill point 162 allows the bone screw 160 to self drill into a bone, thereby eliminating the additional step of a pilot hole. The double flutes 166 extend to the cutting section edges 172 and into the opening of the lumen passage 170. Bone chips cut by the drill point 162 and thread cutting edges 172 are directed into the lumen passage 170 to fill the elongated body member 164. Additionally, a bone screw 180 is provided having an alternate head design 182, such as a socket head cap screw.

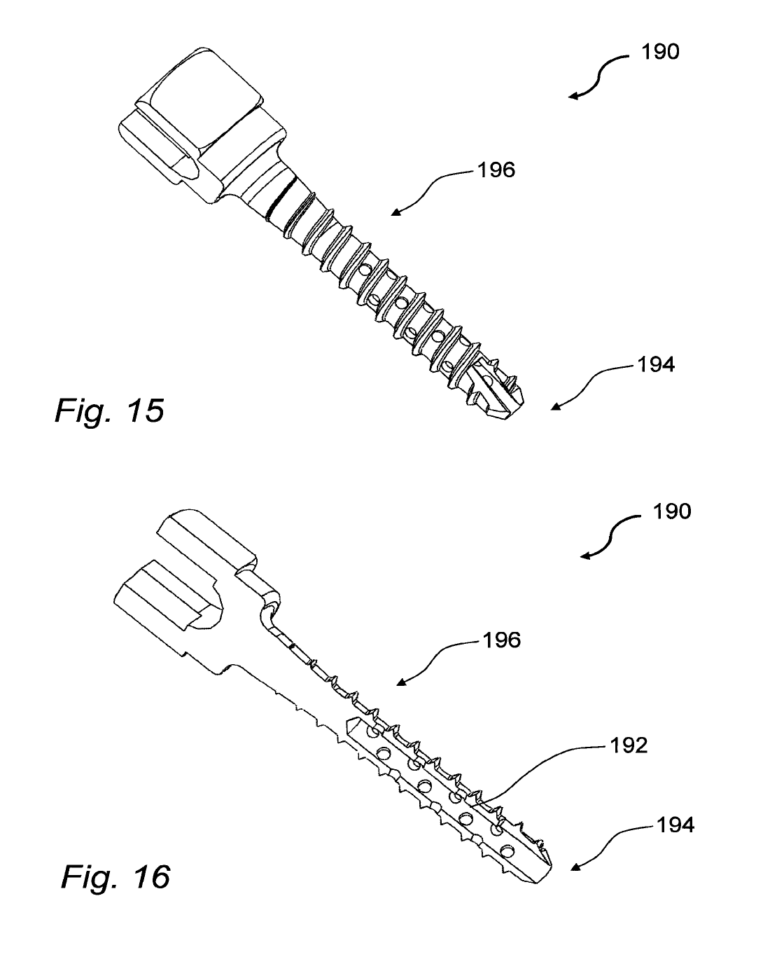

Referring to FIGS. 15 and 16, an alternative embodiment of the self filling autograft bone screw 10 of FIG. 1 is presented. A bone screw 190 is provided which includes a lumen passage 192 starting at a distal end 194 of an elongated body member 196 and ending before reaching a proximal end of the elongated body member 196. In this tulip head pedicle screw style, the proximal end of the elongated body member 196 maintains maximum structural integrity while the distal end 194 incorporates the advantages of the autograft integration of the screw into the bone and the subsequent strength improvement of the surrounding bone structure.

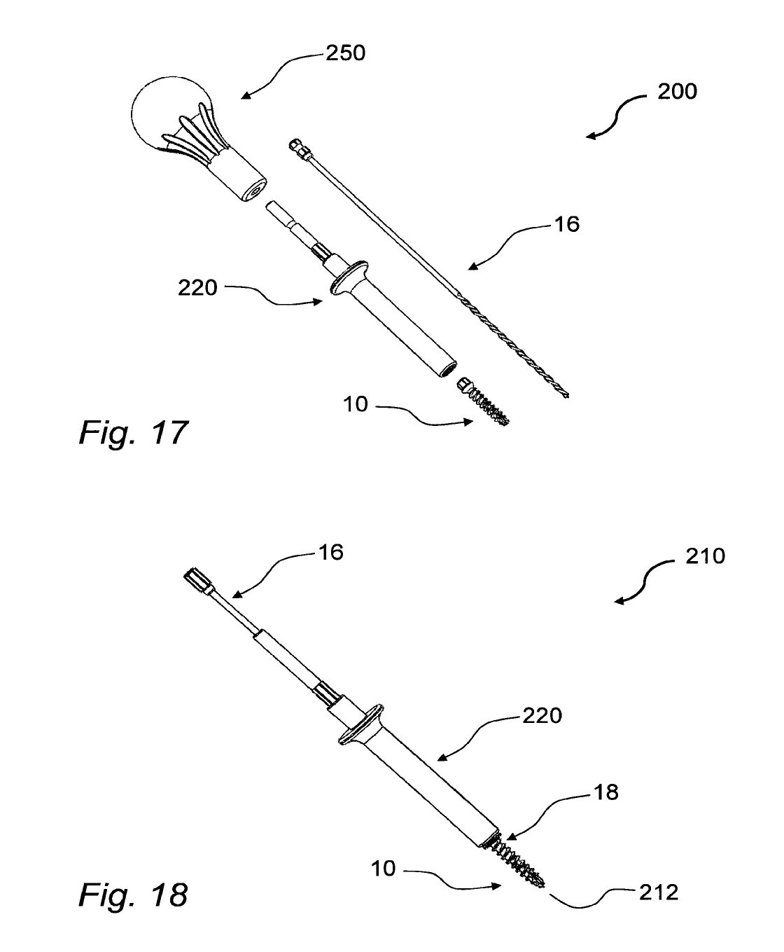

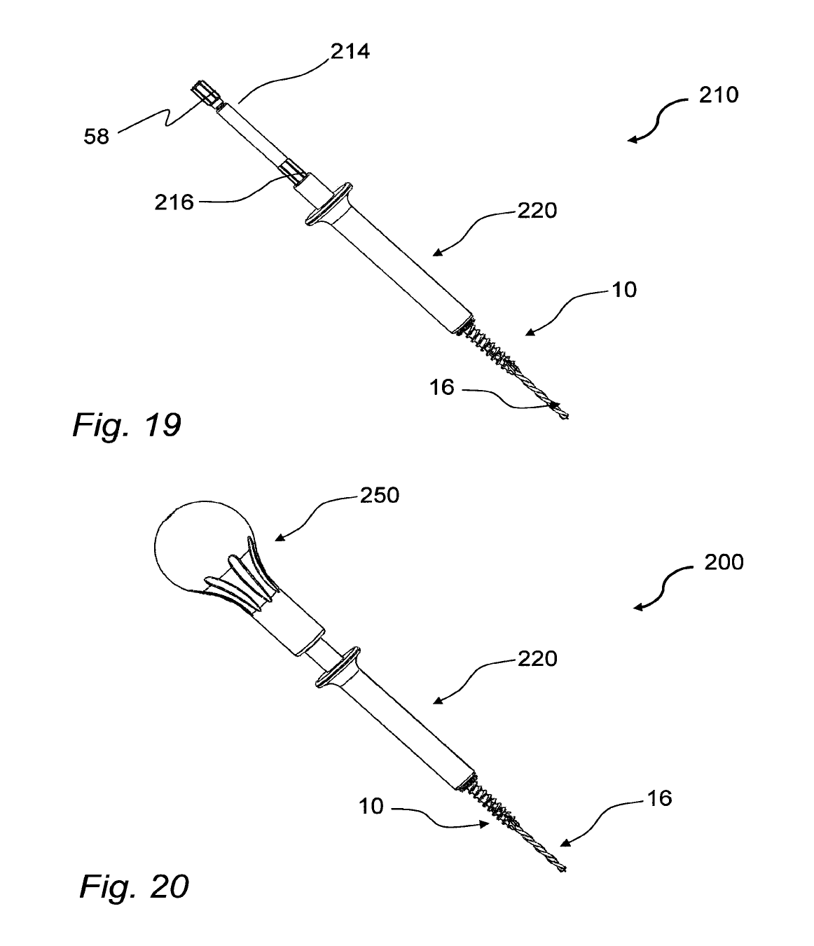

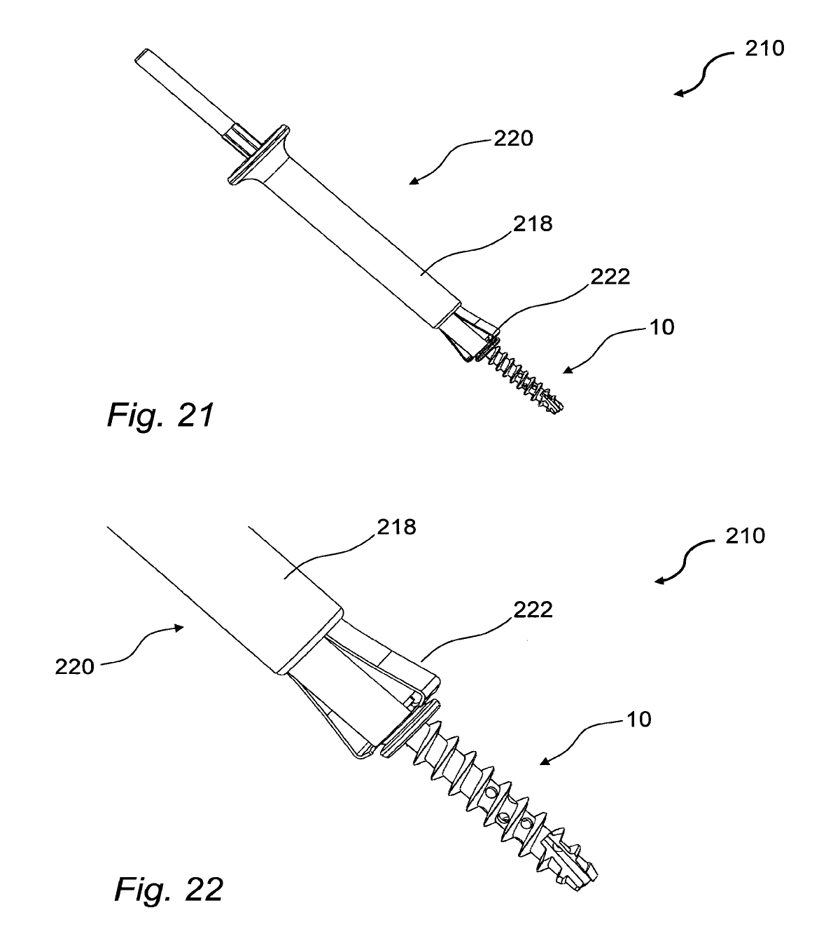

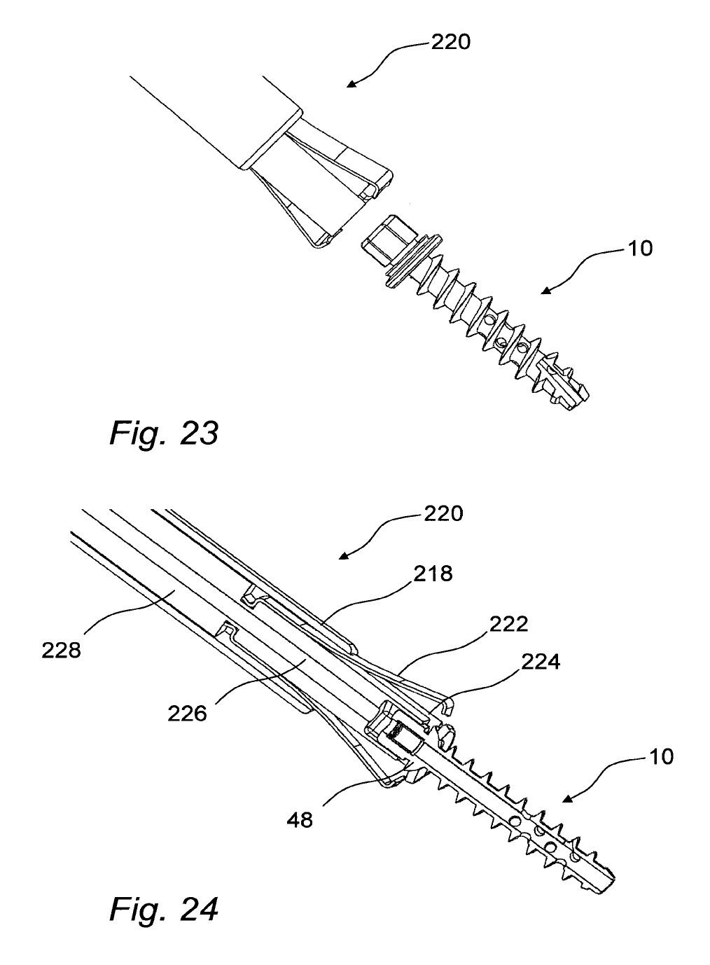

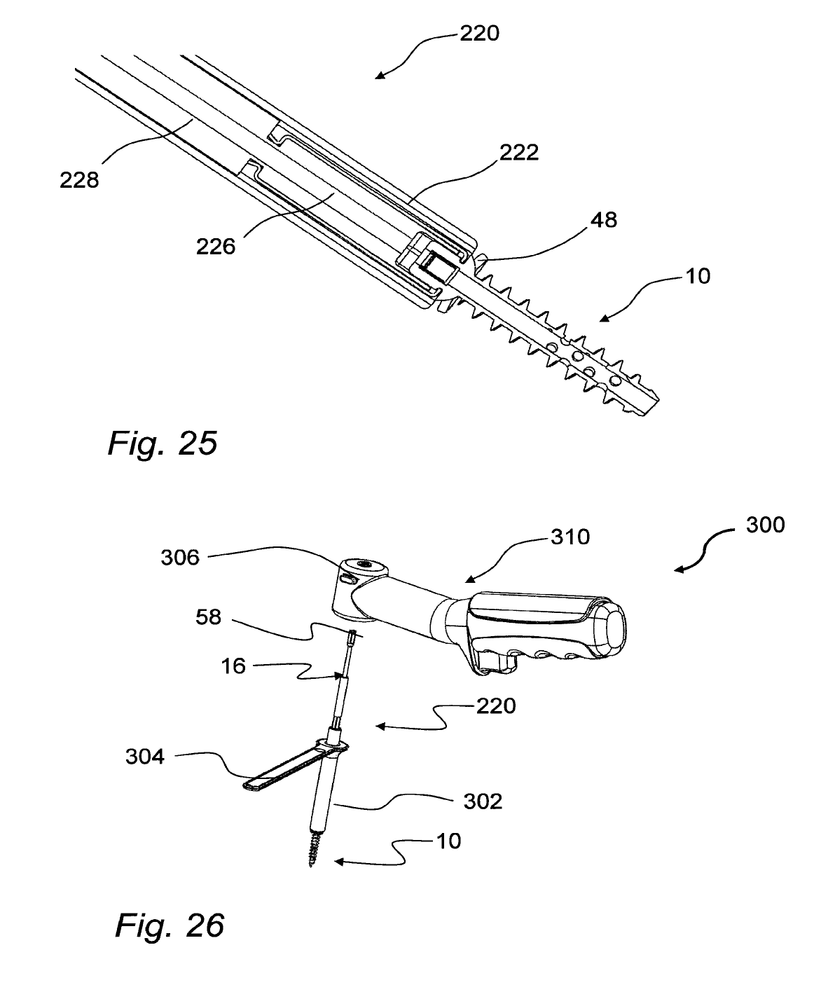

Referring to FIGS. 17 through 25, embodiments of an external fastening member and embodiments of the self filling autograft bone screw 10 of FIG. 1 are presented. In one embodiment, a system 200 is presented that includes an external fastening member 220, a drive handle 250, and a bone screw 10. The system 200 may also include a guide drill 16, although the guide drill 16 may not be required when using a self drilling embodiment of the bone screw 10.

In another embodiment, a system 210 is presented which includes the bone screw 10 positioned within the external fastening member 220 with the guide drill 16 inserted through the cannulated center of the external fastening member 220 until the tip of the guide drill 16 extends past the end 212 of the bone screw 10. Once assembled, the external fastening member 220, bone screw 10, and guide drill 16 may be placed through a small incision to the bone (e.g. pedicle, facet joint, etc). The guide drill may then be advanced into the bone until a depth stop 214 on the guide drill 16 contacts the mating surface on the external fastening member 220. The guide drill 16 may be advanced by applying torque and axial force through the drive structure 58 of the guide drill 16. This can be accomplished using the manual drive handle 250 or some form of powered drill type device.

In another embodiment, the drive handle 250 is engaged with the external fastening member 220. The drive handle 250 advances the bone screw 10 into the bone while the guide drill 16 remains stationary, thereby providing a guide for the proper positioning of the bone screw 10. Once inserted, the bone screw 10 is released from the external fastening member 220 by sliding a sleeve 218 axially along the external fastening member 220 towards the proximal end of the bone screw 10. The act of sliding the sleeve 218 also uncovers spring catches 222 and allows the spring catches 222 to deflect outward to their normal position. The ends of the spring catches 222 are held into the undercut features 48 of the bone screw 10 by the sleeve 218, which prevents the spring catches 222 from opening outward. When the sleeve 218 is slid axially away, the spring catches 222 are free to deflect out of the undercuts 48. The cannulated opening 226 on the drive shaft 228 allows the guide drill 16 to pass through it. The external fastening member 220 may now be removed from the patient's body.

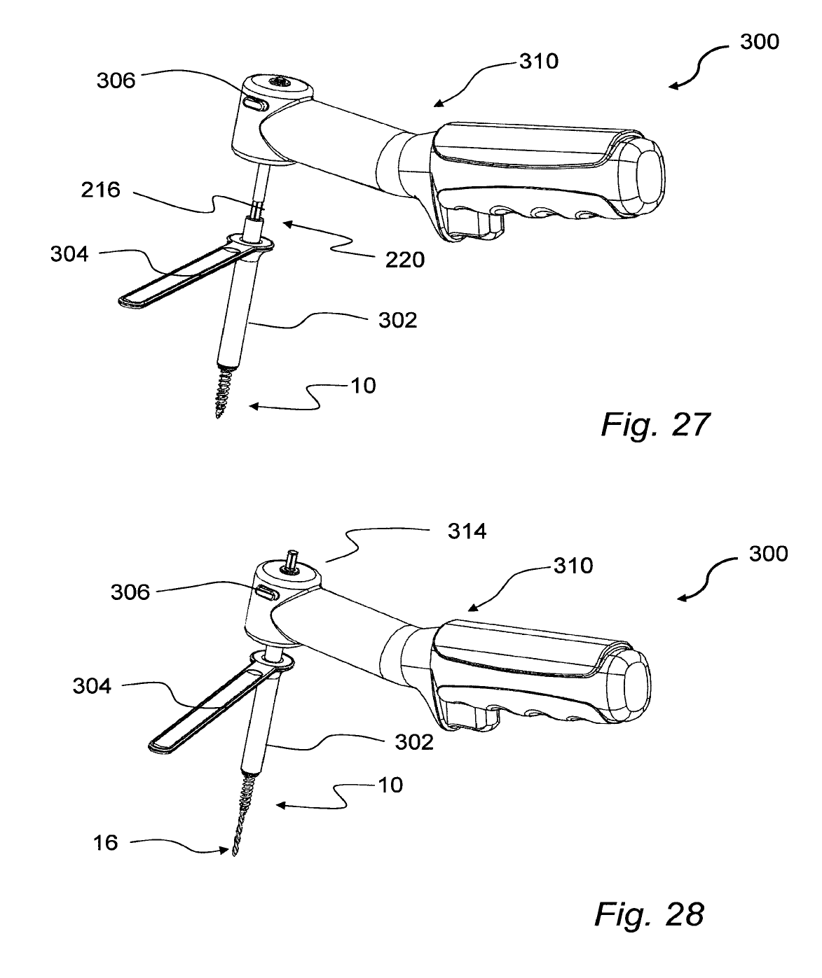

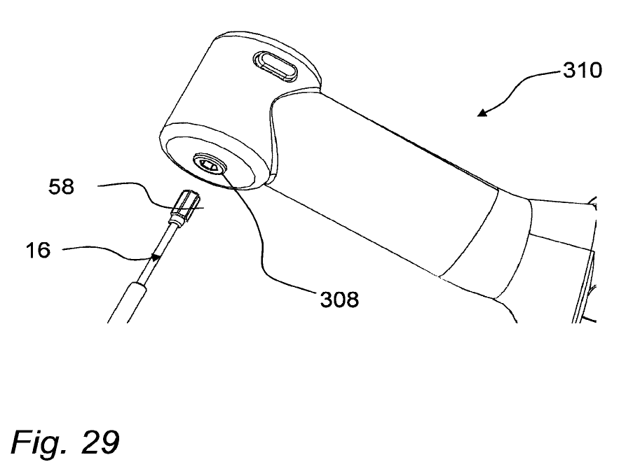

Referring to FIGS. 26 through 29, an alternative embodiment of an external fastening member and the self filling autograft bone screw 10 of FIG. 1 are presented. An external fastening member 300 is presented which can be used in place of the manual drive handle 250 and fastening member 220.

In one embodiment, a radio-lucent, off-angle drill 310 is used to provide percutaneous insertion of the bone screw 10 using image guidance. A sleeve 302 with a handle 304 is provided as a means to stabilize the assembly while keeping an operator's hands out of the fluoroscope radiation beam during the image guidance.

The drill 310 captures the drive structure 58 of the guide drill 16. The drill 310 may be powered such that pressure is applied to advance the guide drill 16 into the bone. The drill 310 engages the drive structure 216 of the insertion tool 220. The drill 310 can advance the bone screw 10 under power into the bone. The guide drill 16 is removed by pulling it fully through the drill upper surface 314. The sleeve 302 may be slid axially to release the bone screw 10. Once the bone screw 10 is advanced into the bone, the guide drill 300 may release the drive structure 58 of the guide drill 16 by pressing the release button 306.

It will be understood that various modifications may be made to the embodiments disclosed herein. Therefore, the above description should not be construed as limiting, but merely as illustrative of some embodiments according to the invention.

What is claimed is:

1. A self-filling autograft bone screw, comprising:
an elongated body member having a proximal portion and a distal portion;
a lumen disposed within the elongated body member, the lumen extending from a proximal end of the elongated body member to a distal end of the elongated body member;
a plurality of external threads extending from the proximal portion of the elongated body member to the distal portion of the elongated body member, the plurality of external threads adapted for anchoring the elongated body member within an internal portion of a bone within a patient's body;
a cutting section disposed at the distal end of the elongated body member, the cutting section adapted to enable penetration of the bone screw into the internal portion of the bone and facilitate the insertion of fragments into the lumen resulting from the penetration of the bone screw into the internal portion of the bone; and
at least one opening disposed along the length of the elongated body member, the at least one opening adapted for facilitating the re-growth of the fragments within the internal portion of the bone and anchoring of the elongated body member within the internal portion of the bone.

2. The bone screw of claim 1, wherein the cutting section has a helically-shaped configuration.

3. The bone screw of claim 1, wherein the cutting section is disposed at an acute angle relative to the longitudinal axis of the elongated body member.

4. The bone screw of claim 1, further comprising a plurality of openings disposed along the length of the elongated body member.

5. The bone screw of claim 1, further comprising a second cutting section disposed proximal to the cutting section adapted to further enable penetration of the bone screw into the internal portion of the bone and to facilitate the insertion of fragments into the lumen resulting from the penetration of the bone screw into the internal portion of the bone.

6. The bone screw of claim 1, wherein the lumen is adapted to engage an external fastening member used to facilitate insertion of the bone screw into the bone within the patient's body.

7. The bone screw of claim 6, wherein the external fastening member is a drill.

8. The bone screw of claim 6, wherein the external fastening member is a ratchet.

9. The bone screw of claim 1, wherein the fragments include any one or more of bone chips, bone marrow, and blood.

10. The bone screw of claim 1, wherein the proximal portion of the elongated body member has a diameter that is greater than a diameter of the distal portion of the elongated body.

11. The bone screw of claim 1, wherein the proximal portion of the elongated body member has a diameter that is equal to a diameter of the distal portion of the elongated body.

12. The bone screw of claim 1, wherein the proximal portion includes a drive structure adapted for transmitting torque to facilitate insertion of the bone screw into the bone.

13. The bone screw of claim 1, wherein the proximal portion includes a washer member adapted for pivoting on the proximal end of the bone screw to contact with an angled surface of the bone.

14. A screw system for inserting a bone screw into a bone of a patient's body, comprising:
an external fastening member used to facilitate insertion of the self filling bone screw into the bone within the patient's body, the external fastening member including at least one flute member disposed along the length of the external fastening member;
the self filling bone screw having an elongated body member having a proximal portion and a distal portion;
a lumen disposed within the elongated body member, the lumen extending from a proximal end of the elongated body member to a distal end of the elongated body member;
a plurality of external threads extending from the proximal portion to the distal portion, the plurality of external threads adapted for anchoring the elongated body member within an internal portion of a bone within a patient's body;
a cutting section disposed at the distal end of the elongated body member, the cutting section adapted to enable penetration of the bone screw into the internal portion of the bone and facilitate the insertion of fragments into the lumen resulting from the penetration of the bone screw into the internal portion of the bone; and
at least one opening disposed along the length of the elongated body member, the at least one opening adapted for facilitating the re-growth of the fragments within the internal portion of the bone and anchoring of the elongated body member within the internal portion of the bone.

15. The system of claim 14, wherein the at least one flute member is adapted for capturing the fragments resulting from the penetration of bone screw into the bone.

16. The system of claim 14, wherein the bone screw includes a wiper member adapted for preventing the fragments from dispersing externally from the lumen.

17. A method for inserting a bone screw into a bone of a patient's body, comprising:
providing a self-filling bone screw having an elongated body member having a proximal portion and a distal portion; a lumen disposed within the elongated body member, the lumen extending from a proximal end of the elongated body member to a distal end of the elongated body member; a plurality of external threads extending from the proximal portion to the distal portion, the plurality of external threads adapted for anchoring the elongated body member within an internal portion of a bone within a patient's body; a cutting section disposed at the distal end of the elongated body member, the cutting section adapted to enable penetration of the bone screw into the internal portion of the bone and facilitate the insertion of fragments into the lumen resulting from the penetration of the bone screw into the internal portion of the bone; at least one opening disposed along the length of the elongated body member, the at least one opening adapted for facilitating the re-growth of the fragments within the internal portion of the bone and anchoring of the elongated body member within the internal portion of the bone;
forming a hole within the bone;
advancing the bone screw into the hole within the bone; and
positioning the bone screw into the hole within the bone.

18. The method of claim 17, wherein the bone screw is advanced through an external fastening member.

19. The method of claim 18, wherein the external fastening member is a drill.

20. The method of claim 17, wherein the external fastening member is a ratchet.

21. The method of claim 17, further comprising extracting the external fastening member from the hole within the bone.
